(19) United States
(12) Patent Application Publication
(10) Pub. No.: US 2007/0105816 A1
(43) Pub. Date: May 10, 2007

(54) Edulcorant compositions

(75) Inventor: Alessandro Seneci, Segrate (IT)

Correspondence Address: Stephen M Nipper;Dykas Shaver & Nipper, PO Box 877, Boise, ID, 83701-0877, US

(73) Assignee: STRIP TEASE SAS DI ALLESSANDRO SENECI & C., 20121 MILANO (IT)

(21) Appl. No.: 10/556,241

(22) PCT Filed: May 4, 2004
(86) PCT No.: PCT/IT04/00242

§ 371 Date: Dec. 8, 2006

(30) Foreign Application Priority Data
May 6, 2003 (IT) MI2003A000907

(51) Int. Cl. A61K 31/715; A01N 43/04

(52) U.S. Cl. 514/57

(57) ABSTRACT

An edulcorating composition containing 5 to 20% by weight of inulin or fruitoligosaccharides, from 2 to 15% by weight of jellying fibres, 0.5 to 5% by weight of cellulose and 60 to 92.5% by weight of monosaccharides or disaccharides.

The present invention refers to a new edulcorating composition for food and/or dietetic use in order to reduce the glycemic peak due to the swallowing of monosaccharides or disaccharides with a edulcorating effect; it is also able to bring soluble fibres with a prebiotic affect useful for the re-equilibrium and the feeding of the bacterial flora.

The composition according to the present invention contains 5 to 20% by weight of inulin or fruitoligosaccharides, 0.5 to 5% by weight of cellulose and 60 to 92.5% of monosaccharides or disaccharides, with respect to the whole weight of such composition (i.e. where the sum of the single components is 100).

The jellying fibre is selected preferably from glucomannano, guar gum, caraja gum or pectin; the monosaccharides are preferably selected from fructose and/or dextrose; the disaccharides are preferably selected from saccharose, lactose and/or maltose; the cellulose is preferably in a microcrystalline form.

The jellying fibre is selected preferably from glucomannano, guar gum, caraja gum or pectin; the monosaccharides are preferably selected from fructose and/or dextrose; the disaccharides are preferably selected from saccharose, lactose and/or maltose; the cellulose is preferably in a microcrystalline form.

According to a first preferred embodiment of the invention, the composition contains 7 to 18% by weight of inulin or fruitoligosaccharides, preferably 10 to 16% by weight, more preferably about 15% by weight.

According to a second embodiment of the invention, the composition contains 3 to 10% by weight of jellying fibres, preferably 3.5 to 8% by weight, more preferably about 4% by weight.

According to a third preferred embodiment of the invention, the composition contains 0.8 to 3% by weight of cellulose, preferably 1% by weight

According to a last preferred embodiment, the composition contains 70 to 90% by weight of monosaccharides or disaccharides, preferably 75 to 85% by weight, more preferably about 80% by weight.

The edulcorating composition of the present invention can be prepared by simply mixing of the single components, by dry granulating (pre-compression) or by wet on a fluid bed, according to techniques well known in the art.

The object of the present invention is not limited to the edulcorating compositions above described but also to the use of the single components, in the above mentioned ratio, for the preparation of edulcorating compositions able to reduce the glycemic and/or insulin peak due to the swallowing of monosaccharides and/or disaccharides in an amount equal or equivalent to that contained in the edulcorating composition according to the invention, as clearly showed in the following example, and which has to be considered as illustrative and not limiting of the same.

### EXAMPLE

A edulcorating composition having the following quali-quantitative composition to 12 health volunteers has been administered (T treatnent):

| | |
|---|---|
| saccharose | 60 grams |
| inulin | 12 grams |
| glucomannano(flour of Kanjac) | 2.25 grams |
| microcrystalline cellulose | 0.75 grams |

To the same volunteers 60 grams of saccharose (k treatment) have been administered.

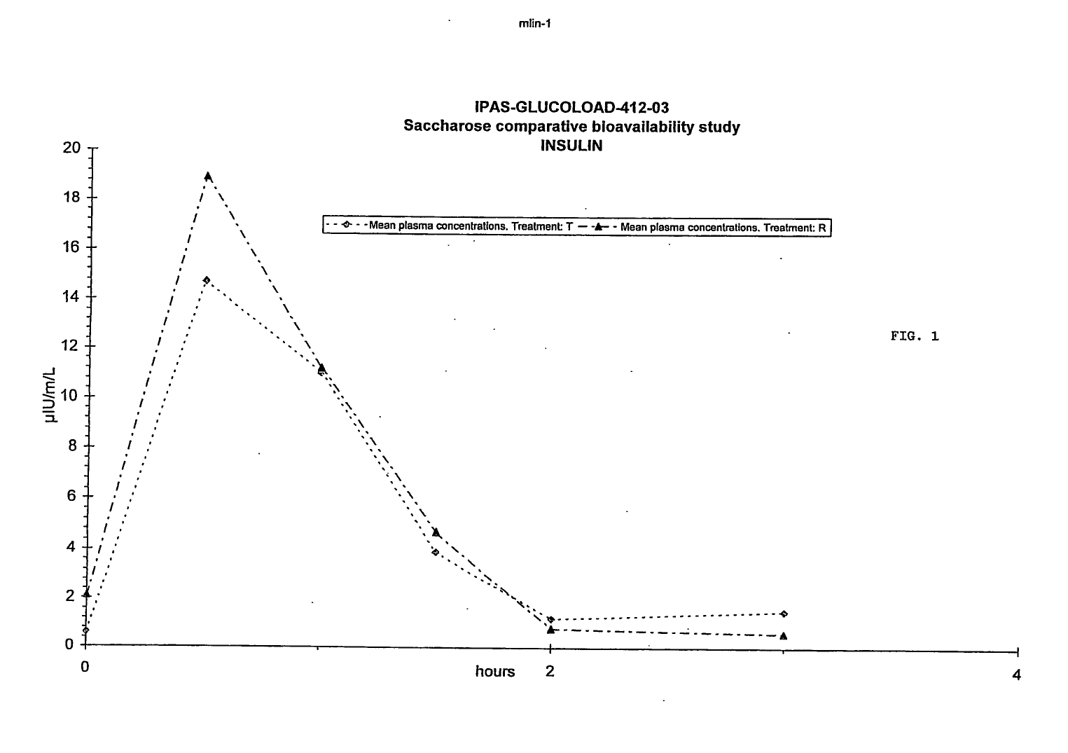

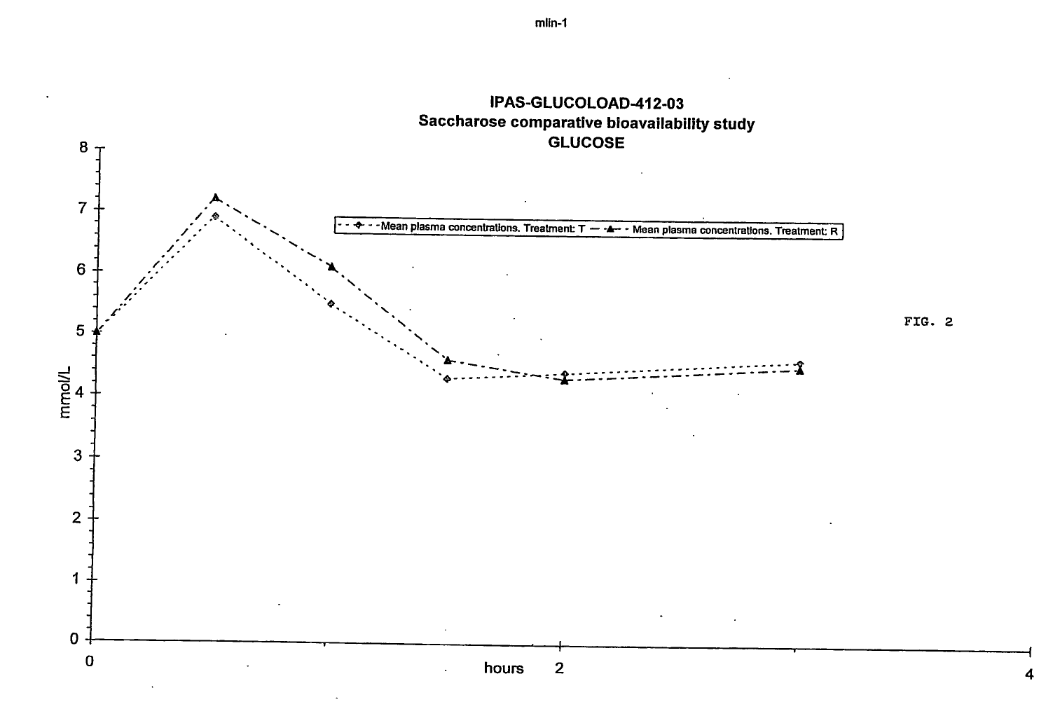

The plasmatic concentration of inulin and glucose due to the aforesaid administrations are shown in FIG. 1 and 2, respectively.

1. Edulcorating composition containing 5 to 20% by weight of inulin or fruit-oligosaccharides, 2 to 15% by weight of jellying fibre, 0.5 to 5% by weight of cellulose and 60 to 92.5 by weight of monosaccharides or disaccharides.

2. Edulcorating composition according to claim 1 characterised in that said jellying fibre is selected from glucomannano, guar gum, caraja gum or pectin.

3. Edulcorating composition according to claim 1, characterised in that such monosaccharides are selected from fructose and/or dextrose.

4. Edulcorating composition according to claim 1, characterised in that such disaccharides are selected from saccharose, lactose and/or maltose.

5. Edulcorating composition according to claim 1, characterised in that the cellulose is in microcrystalline form.

6. Edulcorating composition according to any preceding claims, characterised in that it contains 7 to 18% by weight of inulin or fruitoligosaccharides.

7. Edulcorating composition according to any preceding claims, characterised in that it contains 10 to 16% by weight of inulin or fruitoligosaccharides.

8. Edulcorating composition according to any preceding claims, characterised in that it contains about 15% by weight of inulin or fruitoligosaccharides.

9. Edulcorating composition according to any preceding claims, characterised in that it contains 3 to 10% by weight of jellying fibres.

10. Edulcorating composition according to any preceding claims, characterised in that it contains 3.5% to 8% by weight of jellying fibres.

11. Edulcorating composition according to any preceding claims, characterised in that it contains about 4% by weight of jellying fibres.

12. Edulcorating composition according to any preceding claims, characterised in that it contains 0.8 to 3% by weight of cellulose.

13. Edulcorating composition according to any preceding claims, characterised in that it contains about 1% by weight of cellulose.

14. Edulcorating composition according to any preceding claims, characterised in that it contains 70 to 90% by weight of monosaccharides or disaccharides.

15. Edulcorating composition according to any preceding claims, characterised in that it contains 75 to 85% by weight of monosaccharides or disaccharides.

16. Edulcorating composition according to any preceding claims, characterised in that it contains about 80% by weight of monosaccharides or disaccharides.

17. Edulcorating composition according to any preceding claims, characterised in that it contains about 15% by weight of inulin of fruitoligosaccharides, about 4% by weight of jellying fibres, about 1% by weight of cellulose and about 80% by weight of monosaccharides or disaccharides.
